(19) United States
(12) Patent Application Publication
(10) Pub. No.: US 2026/0183046 A1
(43) Pub. Date: Jul. 2, 2026

(54) TECHNOLOGIES FOR CONTROLLING A COMBINED RADIO FREQUENCY AND ULTRASONIC MODE OF ENERGY-BASED SURGICAL INSTRUMENTS

(71) Applicant: Cilag GmbH International, Zug (CH)

(72) Inventors: Cameron D. McLain, Cincinnati, OH (US); Daniel Boguszewski, Cincinnati, OH (US); Jorge Vélez Martinez, Cincinnati, OH (US); Guion Lucas, Cincinnati, OH (US); Bruce Brunson, JR., Cincinnati, OH (US); Jacob Gee, Cincinnati, OH (US); Monica Rivard, Cincinnati, OH (US); Jacqueline C. Aronhalt, Cincinnati, OH (US)

(21) Appl. No.: 19/341,060

(22) Filed: Sep. 26, 2025

Related U.S. Application Data
(60) Provisional application No. 63/740,944, filed on Dec. 31, 2024.

(51) Int. Cl. A61B 18/14; A61B 18/00

(52) U.S. Cl. CPC A61B 18/1445; A61B 2018/00601; A61B 2018/0063; A61B 2018/00702; A61B 2018/00875; A61B 2018/00994

(57) ABSTRACT

A control method for an energy-based surgical instrument includes activating a combined radio frequency (RF) and ultrasound operation, which may be a sealing operation or a sealing and transection operation. The sealing operation includes activating a pulsed RF operation with an ultrasound sealing operation between pulses of the RF operation. A control method for the energy-based surgical instrument includes determining whether an ultrasonic blade of the surgical instrument contacts a tissue pad of the surgical instrument and, if so, allowing ultrasonic energy delivery at a reduced energy level or preventing ultrasonic energy delivery. Other embodiments are described and claimed.

### CROSS-REFERENCE TO RELATED APPLICATION

This application claims the benefit of and priority to U.S. patent application Ser. No. 63/740,944, entitled “TECHNOLOGIES FOR THERAPEUTIC AND SUBTHERAPEUTIC CONTROL OF ENERGY-BASED SURGICAL INSTRUMENTS,” which was filed on Dec. 31, 2024, and which is incorporated herein by reference in its entirety.

### TECHNICAL FIELD

The present disclosure relates generally to energy-based surgical instruments and, more particularly, to harmonic and/or electrosurgical surgical instruments.

### BACKGROUND

Energy-based surgical instruments are finding increasingly widespread applications in surgical procedures by virtue of their unique performance characteristics. Depending upon specific device configurations and operational parameters, energy-based surgical instruments can provide both transection of tissue and hemostasis of the tissue by coagulation, which may reduce or otherwise minimize patient trauma. Depending on the particular application, energy-based surgical instruments may utilize different surgical technologies including, for example, ultrasonic and/or electro-surgical (e.g., radio frequency (RF)) technologies.

A typical ultrasonic surgical instrument may include a handpiece containing an ultrasonic transducer and an elongated shaft assembly having a distally mounted end effector to effect the cutting and sealing of tissue. For example, the end effector may include a jaw assembly having an ultrasonic blade and a clamp arm, which may include a non-stick tissue pad or similar bed to receive the ultrasonic blade. In some cases, the elongated shaft assembly may be permanently affixed to the handpiece. In other cases, the elongated shaft assembly may be detachable from the handpiece, as in the case of a disposable shaft assembly or a shaft assembly that is interchangeable between different handpieces. In use, the end effector transmits ultrasonic energy to tissue brought into contact with the ultrasonic blade of the end effector to realize the cutting and sealing action. Such ultrasonic surgical devices may be configured for open surgical use, laparoscopic, and/or endoscopic surgical procedures including robotic-assisted procedures.

Ultrasonic energy cuts and coagulates tissue using temperatures lower than those used in electro-surgical procedures. Vibrating at high frequencies (e.g., 55,500 times per second), the ultrasonic blade denatures protein in the tissue to form a sticky coagulum. Pressure exerted on tissue by the ultrasonic blade surface collapses blood vessels and allows the coagulum to form a hemostatic seal. A surgeon can control the cutting speed and coagulation by the force applied to the tissue by the end effector, the time over which the force is applied, and the selected excursion level of the end effector.

In electro-surgical instruments, one or more electrodes are incorporated into the end effector and configured to apply therapeutic electrical current to the patient's tissue to create a hemostatic seal. In electro-surgical instruments that do not include a harmonic mode (i.e., do not include a harmonic blade), the end effector may be embodied as two clamp arms or jaws. In such embodiments, the electro-surgical instrument may include a separate mechanical knife or blade for cutting the tissue after the creation of the hemostatic seal, which may be incorporated into the elongated shaft attached to the end effector. In bi-polar embodiments, an active electrode may be attached to one of the clamp arms of the end effector and configured to introduce an electrical current into the tissue, which is received by a return electrode attached to the other clamp arm of the end effector (or as the blade itself in embodiments including a harmonic mode). Conversely, in mono-polar embodiments, the return electrode (e.g., a “grounding pad”) may be separate from the electro-surgical instrument and located on a different part of the body of the patient. In some embodiments, the electro-surgical instrument may also be configured to apply a sub-therapeutic electrical current to the patient's tissue, which may be used for sensing purposes (e.g., measuring tissue impedance).

Electro-surgery forms hemostatic seals by generating heat in the tissue via the introduced electrical energy, which is embodied as radio frequency (“RF”) energy. The particular frequency employed can vary based on the intended use of the electro-surgical instrument within the range of about 100 kHz to 1 MHz, although higher frequencies can be employed in some embodiments. Additionally, sub-therapeutic frequencies may be used in some situations for purposes other than hemostatic sealing, such as performing various electrical measurements on the tissue.

It should be appreciated that some energy-based surgical instruments may employ dual or multi-modal technologies for the transection and/or hemostasis of patient tissue. For example, in some cases, an energy-based surgical instrument may include both ultrasonic and electro-surgical capabilities (e.g., by utilizing the ultrasonic blade as an electrode for the electro-surgery mode), which increases the surgical options provided by the surgical instrument to the surgeon.

### SUMMARY

According to an aspect of the present disclosure, a method for controlling a surgical instrument includes activating, by a control element, a pulsed radio frequency (RF) sealing operation with an electrode of an end effector of the surgical instrument, wherein the pulsed RF sealing operation delivers RF energy in a series of pulses; and activating, by the control element, an ultrasound sealing operation with an ultrasonic blade of the end effector between pulses of the pulsed RF sealing operation, wherein the ultrasound sealing operation delivers ultrasonic energy at a first energy level. In some embodiments, the first energy level comprises a sealing energy level.

In some embodiments, the method further includes activating, by the control element, ultrasonic energy at a second energy level with the ultrasonic blade during activation of the pulsed RF sealing operation, wherein the second energy level is lower than the first energy level.

In some embodiments, the method further includes activating, by the control element, ultrasonic energy at a second energy level with the ultrasonic blade before activation of the pulsed RF sealing operation to preheat tissue to a predetermined temperature. In some embodiments, the predetermined temperature comprises 120° C. In some embodiments, the method further includes deactivating, by the control element, the ultrasonic energy during activation of the pulsed RF sealing operation. In some embodiments, the method further includes activating, by the control element, ultrasonic energy at a third energy level with the ultrasonic blade during activation of the pulsed RF sealing operation and after preheating of the tissue, wherein the third energy level is lower than the first energy level.

In some embodiments, the method further includes determining, by the control element, a requested combined RF and ultrasound operation, wherein the requested combined RF and ultrasound operation comprises a seal operation or a seal and transection operation; and performing, by the control element, the requested combined RF and ultrasound operation with the surgical instrument; wherein when the requested combined RF and ultrasound operation comprises the seal operation, performing the requested combined RF and ultrasound operation with the surgical instrument includes activating the pulsed RF sealing operation and activating the ultrasound sealing operation. In some embodiments, when the requested combined RF and ultrasound operation comprises the seal and transect operation, performing the requested combined RF and ultrasound operation with the surgical instrument includes activating, by the control element, an RF seal and transection operation with the electrode of the end effector, wherein the RF seal and transection operation delivers RF energy to tissue of a patient; measuring, by the control element, a tissue impedance while activating the RF seal and transection operation; determining, by the control element, whether the tissue impedance exceeds a predetermined threshold impedance; and activating, by the control element, an ultrasound transection operation with the ultrasonic blade of the end effector in response to determining that the tissue impedance exceeds the predetermined threshold, wherein the ultrasound transection operation delivers ultrasonic energy to the tissue of the patient. In some embodiments, the predetermined threshold impedance comprises 200 Ω. In some embodiments, the method further includes deactivating, by the control element, the RF seal and transection operation in response to activating the ultrasound transection operation. In some embodiments, the method further includes continuing, by the control element, the RF seal and transection operation in response to activating the ultrasound transection operation.

According to another aspect, a method for controlling a surgical instrument includes activating, by a control element, a radio frequency (RF) seal and transection operation with an electrode of an end effector of the surgical instrument, wherein the RF seal and transection operation delivers RF energy to tissue of a patient; measuring, by the control element, a tissue impedance while activating the RF seal and transection operation; determining, by the control element, whether the tissue impedance exceeds a predetermined threshold impedance; and activating, by the control element, an ultrasound transection operation with an ultrasonic blade of the end effector in response to determining that the tissue impedance exceeds the predetermined threshold, wherein the ultrasound transection operation delivers ultrasonic energy to the tissue of the patient. In some embodiments, the predetermined threshold impedance comprises 200 Ω.

In some embodiments, the method further includes deactivating, by the control element, the RF seal and transection operation in response to activating the ultrasound transection operation. In some embodiments, the method further includes continuing, by the control element, the RF seal and transection operation in response to activating the ultrasound transection operation.

According to another aspect, a method for controlling a surgical instrument includes determining, by a control element, whether an ultrasonic blade of an end effector of the surgical instrument is contacting a tissue pad of the end effector of the surgical instrument; allowing, by the control element, ultrasonic energy delivery at a first energy level in response to determining that the ultrasonic blade is not contacting the tissue pad; and allowing, by the control element, ultrasonic energy delivery at a second energy level lower than the first energy level in response to determining that the ultrasonic blade is contacting the tissue pad.

In some embodiments, the method further includes determining, by the control element, whether a closure switch of the surgical instrument indicates that a jaw clamp of the end effector is closed; wherein allowing the ultrasonic energy delivery at the first energy level further includes allowing the ultrasonic energy delivery at the first energy level in response to determining that the closure switch indicates that the jaw clamp is not closed or determining that the ultrasonic blade is not contacting the tissue pad; and wherein allowing the ultrasonic energy delivery at the second energy level includes allowing the ultrasonic energy delivery at the second energy level in response to determining that the closure switch indicates that the jaw clamp is closed and determining that the ultrasonic blade is contacting the tissue pad.

In some embodiments, the method further includes indicating, by the control element, that the ultrasonic blade is contacting the tissue pad in response to determining that the ultrasonic blade is contacting the tissue pad.

In some embodiments, determining whether the ultrasonic blade is contacting the tissue pad includes applying subtherapeutic radio frequency (RF) energy with an electrode of the end effector; measuring electrical impedance while applying the subtherapeutic RF energy; and determining whether the electrical impedance exceeds a first threshold, wherein the first threshold is indicative of an open circuit.

In some embodiments, determining whether the ultrasonic blade is contacting the tissue pad includes applying therapeutic or subtherapeutic ultrasonic energy with the ultrasonic blade; measuring ultrasonic impedance or ultrasonic impedance slope while applying the therapeutic or subtherapeutic ultrasonic energy; and determining whether the ultrasonic impedance or ultrasonic impedance slope exceeds a positive threshold after the ultrasonic impedance has reduced.

In some embodiments, determining whether the ultrasonic blade is contacting the tissue pad includes monitoring jaw aperture data, monitoring force on the jaws, or monitoring vision system data. In some embodiments, determining whether the ultrasonic blade is contacting the tissue pad includes monitoring a plurality of data sources, wherein the plurality of data sources comprises a subtherapeutic RF signal, a subtherapeutic ultrasound signal, a therapeutic ultrasound signal, jaw aperture data, jaw force data, or vision system data.

In some embodiments, allowing the ultrasonic energy delivery at the second energy level includes reducing ultrasound energy delivery to the ultrasonic blade from the first level to the second level in response to determining that the ultrasonic blade is contacting the tissue pad. In some embodiments, the method further includes waiting, by the control element, a predetermined time after reducing the ultrasound energy delivery, wherein the predetermined time corresponds to a predetermined user reaction time; and stopping, by the control element, ultrasound energy delivery after waiting the predetermined time.

In some embodiments, the second energy level comprises a zero level, and allowing the ultrasonic energy delivery at the second energy level includes preventing ultrasonic energy delivery. In some embodiments, the method further includes receiving, by the control element, a command to activate the ultrasonic energy delivery to the ultrasonic blade of the end effector; wherein determining whether the ultrasonic blade is contacting the tissue pad includes determining whether the ultrasonic blade is contacting the tissue pad in response to receiving the command to activate the ultrasonic energy delivery.

According to another aspect, a system for controlling a surgical instrument includes the surgical instrument and a control element. The surgical instrument comprises an end effector having an electrode configured to deliver radio frequency (RF) energy to tissue of a patient and an ultrasonic blade configured to delivery ultrasonic energy to the tissue of the patient. The control element is configured to activate a pulsed RF sealing operation with the electrode of the surgical instrument, wherein the pulsed RF sealing operation delivers RF energy in a series of pulses, and activate an ultrasound sealing operation with the ultrasonic blade of surgical instrument between pulses of the pulsed RF sealing operation, wherein the ultrasound sealing operation delivers ultrasonic energy at a first energy level. In some embodiments, the first energy level comprises a sealing energy level.

In some embodiments, the control element is further configured to activate ultrasonic energy at a second energy level with the ultrasonic blade during activation of the pulsed RF sealing operation. The second energy level is lower than the first energy level.

In some embodiments, the control element is further configured to activate ultrasonic energy at a second energy level with the ultrasonic blade before activation of the pulsed RF sealing operation to preheat tissue to a predetermined temperature. In some embodiments, the predetermined temperature comprises 120° C. In some embodiments, the control element is further configured to deactivate the ultrasonic energy during activation of the pulsed RF sealing operation. In some embodiments, the control element is further configured to activate ultrasonic energy at a third energy level with the ultrasonic blade during activation of the pulsed RF sealing operation and after preheating of the tissue. The third energy level is lower than the first energy level.

In some embodiments, the control element is further configured to determine a requested combined RF and ultrasound operation and perform the requested combined RF and ultrasound operation with the surgical instrument. The requested combined RF and ultrasound operation comprises a seal operation or a seal and transection operation. When the requested combined RF and ultrasound operation comprises the seal operation, to perform the requested combined RF and ultrasound operation with the surgical instrument includes to activate the pulsed RF sealing operation and activate the ultrasound sealing operation. In some embodiments, when the requested combined RF and ultrasound operation comprises the seal and transect operation, to perform the requested combined RF and ultrasound operation with the surgical instrument includes to activate an RF seal and transection operation with the electrode of the end effector, wherein the RF seal and transection operation delivers RF energy to tissue of a patient; measure a tissue impedance during activation of the RF seal and transection operation; determine whether the tissue impedance exceeds a predetermined threshold impedance; and activate an ultrasound transection operation with the ultrasonic blade of the end effector in response to a determination that the tissue impedance exceeds the predetermined threshold, wherein the ultrasound transection operation delivers ultrasonic energy to the tissue of the patient. In some embodiments, the predetermined threshold impedance comprises 200 Ω. In some embodiments, the control element is further configured to deactivate the RF seal and transection operation in response to activation of the ultrasound transection operation. In some embodiments, the control element is further configured to continue the RF seal and transection operation in response to activation of the ultrasound transection operation.

According to another aspect, a system for controlling a surgical instrument includes the surgical instrument and a control element. The surgical instrument comprises an end effector having an electrode configured to deliver radio frequency (RF) energy to tissue of a patient and an ultrasonic blade configured to delivery ultrasonic energy to the tissue of the patient. The control element is configured to activate an RF seal and transection operation with the electrode of the surgical instrument, wherein the RF seal and transection operation delivers RF energy to the tissue of the patient, measure a tissue impedance during activation of the RF seal and transection operation, determine whether the tissue impedance exceeds a predetermined threshold impedance, and activate an ultrasound transection operation with the ultrasonic blade of the surgical instrument in response to a determination that the tissue impedance exceeds the predetermined threshold, wherein the ultrasound transection operation delivers ultrasonic energy to the tissue of the patient. In some embodiments, the predetermined threshold impedance comprises 200 Ω.

In some embodiments, the control element is further configured to deactivate the RF seal and transection operation in response to activation of the ultrasound transection operation. In some embodiments, the control element is further configured to continue the RF seal and transection operation in response to activation of the ultrasound transection operation.

According to another aspect, a system for controlling a surgical instrument includes the surgical instrument and a control element. The surgical instrument comprises an end effector having an ultrasonic blade configured to delivery ultrasonic energy to tissue of a patient and a tissue pad coupled to a jaw clamp of the end effector. The control element is configured to determine whether the ultrasonic blade contacts the tissue pad, allow ultrasonic energy delivery at a first energy level in response to a determination that the ultrasonic blade does not contact the tissue pad, and allow ultrasonic energy delivery at a second energy level lower than the first energy level in response to a determination that the ultrasonic blade contacts the tissue pad.

In some embodiments, the surgical instrument further includes a closure switch configured to detect whether the jaw clamp of the end effector is closed. The control element is further configured to determine whether the closure switch indicates that the jaw clamp of the end effector is closed. To allow the ultrasonic energy delivery at the first energy level further includes to allow the ultrasonic energy delivery at the first energy level in response to a determination that the closure switch indicates that the jaw clamp is not closed or the determination that the ultrasonic blade does not contact the tissue pad; and to allow the ultrasonic energy delivery at the second energy level further includes to allow the ultrasonic energy delivery at the second energy level in response to a determination that the closure switch indicates that the jaw clamp is closed and the determination that the ultrasonic blade contacts the tissue pad.

In some embodiments, the control element is further configured to indicate that the ultrasonic blade is contacting the tissue pad in response to the determination that the ultrasonic blade contacts the tissue pad.

In some embodiments, the end effector of the surgical instrument further includes an electrode configured to deliver radio frequency (RF) energy to the tissue of the patient. To determine whether the ultrasonic blade contacts the tissue pad includes to apply subtherapeutic RF energy with the electrode of the end effector, measure electrical impedance during application of the subtherapeutic RF energy, and determine whether the electrical impedance exceeds a first threshold, wherein the first threshold is indicative of an open circuit.

In some embodiments, to determine whether the ultrasonic blade contacts the tissue pad includes to apply therapeutic or subtherapeutic ultrasonic energy with the ultrasonic blade; measure ultrasonic impedance or ultrasonic impedance slope during application of the therapeutic or subtherapeutic ultrasonic energy; and determine whether the ultrasonic impedance or ultrasonic impedance slope exceeds a positive threshold after the ultrasonic impedance has reduced.

In some embodiments, to determine whether the ultrasonic blade contacts the tissue pad includes to monitor jaw aperture data, to monitor force on the jaws, or to monitor vision system data. In some embodiments, to determine whether the ultrasonic blade contacts the tissue pad includes to monitor a plurality of data sources, wherein the plurality of data sources comprises a subtherapeutic RF signal, a subtherapeutic ultrasound signal, a therapeutic ultrasound signal, jaw aperture data, jaw force data, or vision system data.

In some embodiments, to allow the ultrasonic energy delivery at the second energy level includes to reduce ultrasound energy delivery to the ultrasonic blade from the first level to the second level in response to determining that the ultrasonic blade contacts the tissue pad. In some embodiments, the control element is further configured to wait a predetermined time after reduction of the ultrasound energy delivery, wherein the predetermined time corresponds to a predetermined user reaction time; and stop ultrasound energy delivery after a wait of the predetermined time.

In some embodiments, the second energy level comprises a zero level, and to allow the ultrasonic energy delivery at the second energy level includes to prevent ultrasonic energy delivery. In some embodiments, the control element is further configured to receive a command to activate the ultrasonic energy delivery to the ultrasonic blade of the end effector. To determine whether the ultrasonic blade contacts the tissue pad includes to determine whether the ultrasonic blade contacts the tissue pad in response to receipt of the command to activate the ultrasonic energy delivery.

### BRIEF DESCRIPTION OF THE DRAWINGS

The detailed description particularly refers to the following figures, in which:

FIG. 1 is a simplified diagram of an embodiment of a system for performing an energy-based surgical procedure;

FIG. 2 is a perspective view of an embodiment of an energy-based surgical instrument of the system of FIG. 1;

FIG. 3 is a side elevation view of a jaw assembly of an end effector of the surgical instrument of FIG. 2 including an ultrasonic blade and in an open state;

FIG. 4 is a side elevation view of the jaw assembly of the end effector of the surgical instrument of FIG. 2 including an ultrasonic blade and in a closed state;

FIG. 5A is a perspective view of another embodiment of the end effector of the surgical instrument of FIG. 2 including an electrode on a lower jaw clamp of the jaw assembly;

FIG. 5B is a perspective view of another embodiment of the end effector of the surgical instrument of FIG. 2 including two jaw clamps, each having an electrode attached thereto;

FIG. 6 is an exploded view of the surgical instrument of FIG. 2;

FIG. 7 is a block diagram of a control circuit of the surgical instrument of FIG. 2;

FIG. 8 is a simplified flow diagram of at least one method for controlling an energy-based surgical instrument;

FIG. 9 is a simplified flow diagram of a method for controlling a combined energy-based surgical instrument;

FIG. 10 is a chart illustrating operation of the surgical instrument according to the method of FIG. 9; and

FIG. 11 is a simplified flow diagram of at least one method for controlling an ultrasound or combined energy-based surgical instrument with blade on pad detection.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, distal, proximal, et cetera, may be used throughout the specification in reference to the surgical instruments described herein as well as in reference to the patient's natural anatomy. Such terms have well-understood meanings in both the study of anatomy and the field of surgery. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

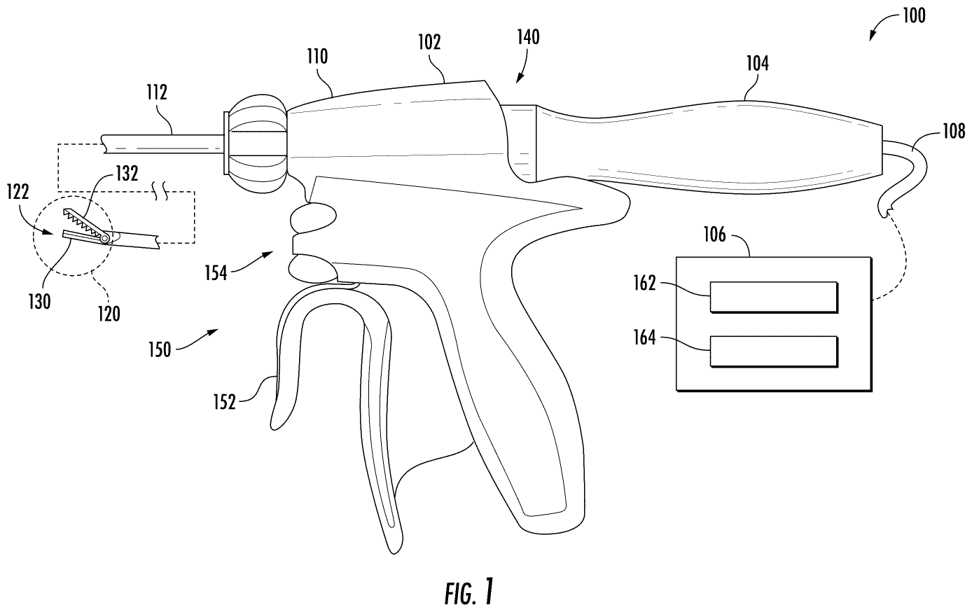

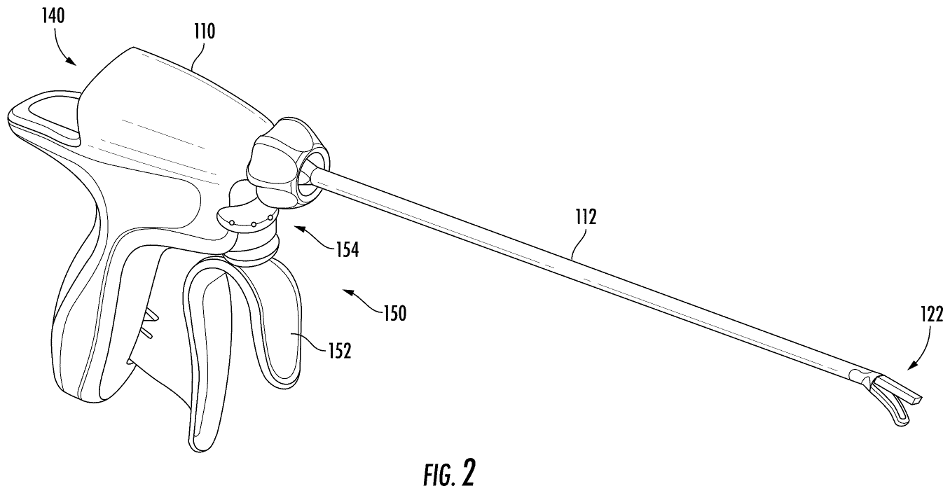

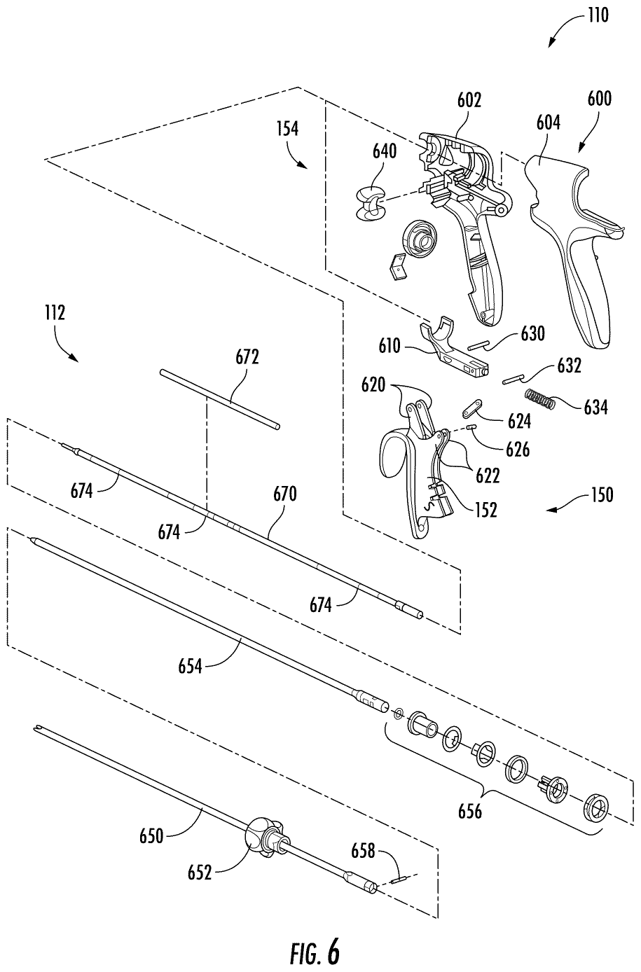

Referring now to FIGS. 1 and 2, in an illustrative embodiment, a system 100 for performing an energy-based surgical procedure includes a surgical instrument 102, a transducer 104, and a generator 106. The surgical instrument 102 is illustratively embodied as an ultrasonic surgical instrument, but may be embodied as an electro-surgical surgical instrument or a multi-modal, ultrasonic/elector-surgical surgical instrument in other embodiments. In use, the surgical instrument 102 is usable to perform various surgical procedures including laparoscopic, endoscopic, or traditional open surgical procedures. In doing so, a surgeon may selectively activate an ultrasonic mode (and/or an electro-surgical/RF mode) of the surgical instrument 102. In the ultrasonic mode, the generator 106 drives the transducer 104 to cause an ultrasonic blade 130 of a jaw assembly 122 of an end effector 120 of the surgical instrument 102 to vibrate at a reference frequency, which facilitates the contemporaneous cutting and hemostatic sealing of patient tissue. Additionally or alternatively, in some embodiments, the surgeon may selectively activate an electro-surgical mode of the surgical instrument 102 to deliver an amount of therapeutic RF energy to the patient tissue to effect hemostatic sealing. In such embodiments, the blade 130 may be embodied as an ultrasonic blade 130 or as a mechanical blade designed to cut tissue using mechanical force (e.g., in those embodiments not employing ultrasonic technologies). Furthermore, in some embodiments, the surgical instrument 102 may be configured with only an electro-surgical/RF mode and, in such embodiments, the jaw assembly 122 of the end effector 120 may not include the ultrasonic blade 130 as discussed in more detail below in regard to FIG. 5B.

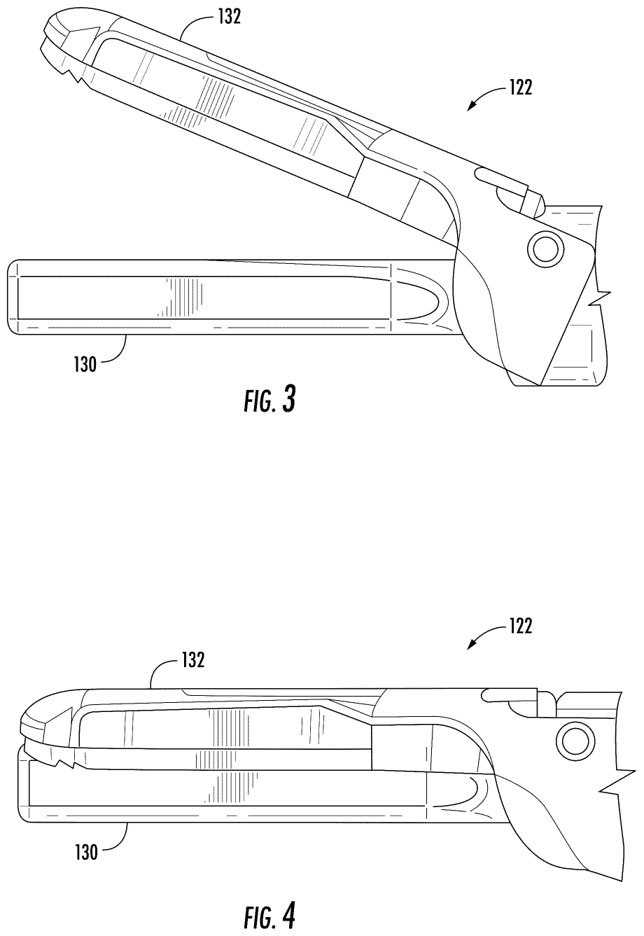

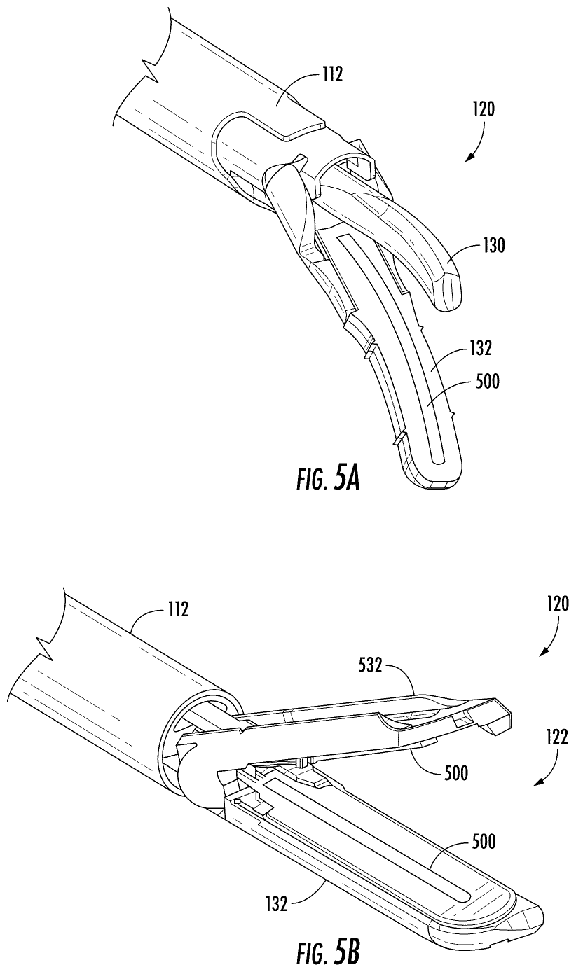

The surgical instrument 102 is illustratively embodied as ultrasonic surgical shears but may be embodied as other types of surgical instruments having an ultrasonic mode and/or electro-surgical mode in other embodiments. In the illustrative embodiment, the surgical instrument 102 includes a handle assembly 110 and an elongated shaft assembly 112, which extends distally away from the handle assembly 110 and may be removably attached to the handle assembly 110 in some embodiments. The elongated shaft assembly 112 includes the end effector 120 located at a distal end opposite the handle assembly 110. The end effector 120 includes the jaw assembly 122, which illustratively includes the ultrasonic blade 130 and a corresponding jaw clamp 132 (but may include two jaw clamps in those embodiments having only an electro-surgical/RF mode). As shown in FIGS. 3 and 4, the jaw assembly 122 is movable between an open state (FIG. 3) in which the jaw clamp 132 is positioned away from the ultrasonic blade 130 and a closed state (FIG. 4) in which the jaw clamp 132 is positioned near or otherwise contacts the ultrasonic blade 130. Actuation of the jaw assembly 122 from the open state to the closed state allows for the grasping, cutting, and coagulation of vessels and/or tissue by the jaw assembly 122. It should be appreciated that the open state may correspond to a degree of openness that is less than a fully opened position of the jaw assembly 122 and the closed state may correspond to a degree of closeness that is less than a fully closed position. That is, the closed state may, for example correspond to a minimal distance between the distal ends of the jaw clamp 132 and the ultrasonic blade 130 and the open state may correspond to a maximum distance between the distal ends of the jaw clamp 132 and the ultrasonic blade 130. However, in other embodiments, the open state may correspond to a fully opened position of the jaw assembly 122 and the closed state may correspond to a fully closed position of the jaw assembly 122.

In those embodiments in which the surgical instrument 102 includes both a ultrasonic mode and an electro-surgical/RF mode, the end effector 120 may include one or more RF electrodes 500 incorporated into the jaw clamp 132 as shown in FIG. 5A. Although the illustrative end effector 120 includes only a single electrode 500 in the embodiment of FIG. 5A, it should be appreciated that the end effector 120 may include additional electrodes 500 in other embodiments (e.g., multiple pads of electrodes 500). The electrode(s) 500 may be embodied as an active electrode configured to the RF energy or as a return electrode configured to “sink” an applied RF energy. In those embodiments utilizing bi-polar RF implementation, the ultrasonic blade 130 may embody the active or return electrode, with the electrode 500 embodying the other active or return electrode. Alternatively, other active or return electrodes may be incorporated on the ultrasonic blade 130 or in another part of the jaw assembly 122 of the end effector 120. In mono-polar implementation, the RF electrode(s) 500 may be embodied as an active electrode, and a return electrode may be attached to a portion of the patient's body.

In those embodiments in which the surgical instrument 102 includes only an electro-surgical/RF mode, the jaw assembly 122 of the end effector 120 includes a jaw clamp 532 in place of the ultrasonic blade 130 as shown in FIG. 5B. In such embodiments, an electrode 500 may be attached to or otherwise incorporated into each jaw clamp 132, 532 and be embodied as an active or a return electrode to facilitate the application of RF energy to tissue captured between the jaw clamps 132, 532. In such embodiments, the surgical instrument 102 may include a knife incorporated into the elongated shaft assembly 112 that is configured to eject outwardly to cut the patient's tissue after sealing of the tissue by the RF energy.

Referring back to FIGS. 1 and 2, in those embodiments including ultrasonic capabilities, the handle assembly 110 includes a receptacle 140 configured to receive the transducer 104 to facilitate connection of the transducer 104 to the handle assembly 110 and the elongated shaft assembly 112. The handle assembly 110 also includes a trigger assembly 150, which includes a primary trigger 152 and a switch assembly 154. The primary trigger 152 is operable by the surgeon to move the jaw assembly 122 of the end effector 120 between the open and closed states. The switch assembly 154 includes one or more buttons, which are selectable by the surgeon to activate (and configure, in some embodiments) the ultrasonic mode and/or the electro-surgical mode of the surgical instrument 102.

The transducer 104 is illustratively connected to the generator 106 by a cable assembly 108. As discussed above, the generator 106 is configured to drive the transducer 104 at a reference or resonant frequency to thereby cause the ultrasonic blade 130 to vibrate. For example, in an illustrative embodiment, the generator 106 may supply an electrical signal to the transducer 104 to cause the ultrasonic blade 130 of the jaw assembly 122 to vibrate longitudinally in the range of, for example, approximately 20 kHz to 250 kHz. In particular embodiments, for example, the ultrasonic blade 130 may vibrate in the range of about 54 kHz to 56 kHz (e.g., at about 55.5 kHz). In other embodiments, the ultrasonic blade 130 may vibrate at other frequencies including, for example, about 31 kHz or about 80 kHz. The excursion of the vibrations at the ultrasonic blade 130 can be controlled by, for example, controlling the amplitude of the electrical signal applied to the transducer 104 by the generator 106. The generator 106 may be activated so that electrical energy may be continuously or intermittently supplied to the transducer 104. The generator 106 also has a power line (not shown) for insertion in an electro-surgical unit or conventional electrical outlet. Additionally or alternatively, the generator 106 may be powered by a direct current (DC) source, such as a battery.

In some embodiments, the generator 106 may be configured to operate in different modes. In such embodiments, the generator 106 may include an ultrasonic generator module 162 for controlling an ultrasonic mode, an electro-surgical/Radio Frequency (RF) generator module 164 for controlling an electro-surgical mode, and/or other generator modules (e.g., a heat generator module) for controlling other operation modes. The various modes of the generator 106 may be operated independently of each other in some embodiments. For example, the generator 106 may activate the ultrasonic mode of the ultrasonic generator module 162 to apply ultrasonic energy to the jaw assembly 122 and subsequently, either therapeutic or sub-therapeutic RF energy may be applied to the jaw assembly 122 by the electro-surgical generator module 164. Alternatively, the activation modes of the generator 106 may be operated simultaneously or contemporaneously with each other.

In the electro-surgical mode, the electro-surgical generator module 164 is configured to generate RF energy at a frequency in the range of about 100 kilohertz (100 kHz) to about 1 megahertz (1 MHz). The generated RF energy is supplied to the patient's tissue via the electrodes 500 of the end effector 120 as described above in regard to FIG. 5. In some embodiments, the electro-surgical generator module 164 may also be configured to selectively provide the RF energy at sub-therapeutic levels to perform various electrical measurements of the patient's tissue. For example, the electro-surgical generator module 164 may be configured to measure an impedance of the patient's tissue using the electrodes 500 and a suitable RF energy level.

Referring now to FIG. 6, as discussed above, the illustrative surgical instrument 102 includes the handle assembly 110 and the elongated shaft assembly 112, which extends distally away from the handle assembly 110. The handle assembly 110 includes a housing 600, which includes a right half-housing 602 and a left half-housing 604. The half-housings 602, 604 are configured to mate with each other to form the housing 600. To facilitate such mating, each of the half-housings 602, 604 may include various interfaces sized to mechanically align and engage one another to form the housing 600 and enclose the internal working components of the surgical instrument 102.

The primary trigger 152 of the trigger assembly 150 is coupled to a linkage mechanism to translate the rotational motion of the primary trigger 152 to axial motion of a yoke 610, which in turn is configured to move the jaw assembly 122 of the end effector 120 between the open and closed states via the elongated shaft assembly 112. The primary trigger 152 includes a first set of flanges 620 having openings formed therein to receive a first yoke pin 630, which extends through the yoke 610. The primary trigger 152 also includes a second set of flanges 622 configured to receive a first end of a link 624. A trigger pin 626 is received in openings formed in the first end of the link 624 and the second set of flanges 622. The trigger pin 626 forms a trigger pivot point for the primary trigger 152. A second end of the link 624, opposite the first end, is received in a slot formed in a proximal end of the yoke 610 and retained therein by a second yoke pin 632. As the primary trigger 152 is rotated about the pivot point formed from the trigger pin 626, the yoke 610 translates horizontally. A spring 634 is used to bias the yoke forward such that the jaw assembly 122 of the end effector 120 is biased to the open state (or a fully opened state).

As discussed above, the trigger assembly 150 also includes a switch assembly 154. The switch assembly 154 illustratively includes a toggle switch 640, which is selectable to activate one or more switches 642. Activation of the switches 642 electrically energizes an electrical element 644, which electrically energizes the ultrasonic transducer 104 to engage the ultrasonic mode of the surgical instrument 102.

The elongated shaft assembly 112 includes an outer tubular sheath 650 and a rotation knob 652 coupled to the outer cylindrical sheath 650. The rotation knob 652 is operable to rotate the outer cylindrical sheath 650 about an axis defined by the outer cylindrical sheath 650. A reciprocating tubular actuator 654 is located within the outer tubular sheath 650 and mechanically engaged with the end effector 120 on a distal end. The reciprocating tubular actuator 654 is also mechanically engaged, on a proximal end, with the yoke 610 within the handle assembly 110 via coupling elements 656. In embodiments including an ultrasonic mode, an ultrasonic waveguide 670 is located within the reciprocating tubular actuator 654. A distal end of the ultrasonic waveguide 670 is acoustically coupled (e.g., directly or indirectly mechanically coupled) to the ultrasonic blade 130, and a proximal end is acoustically coupled to the transducer 104. The ultrasonic waveguide 670 may be isolated from other components of the elongated shaft assembly 112 by a protective sheath 672 and a number of isolation elements 674. The outer tubular sheath 650, the reciprocating tubular actuator 654, and the ultrasonic waveguide 670 are mechanically engaged together via a pin 658.

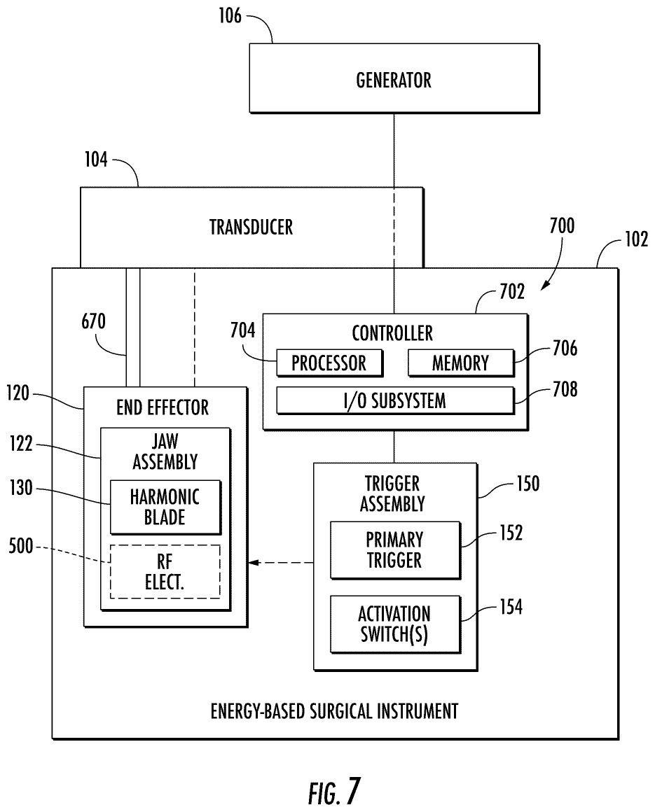

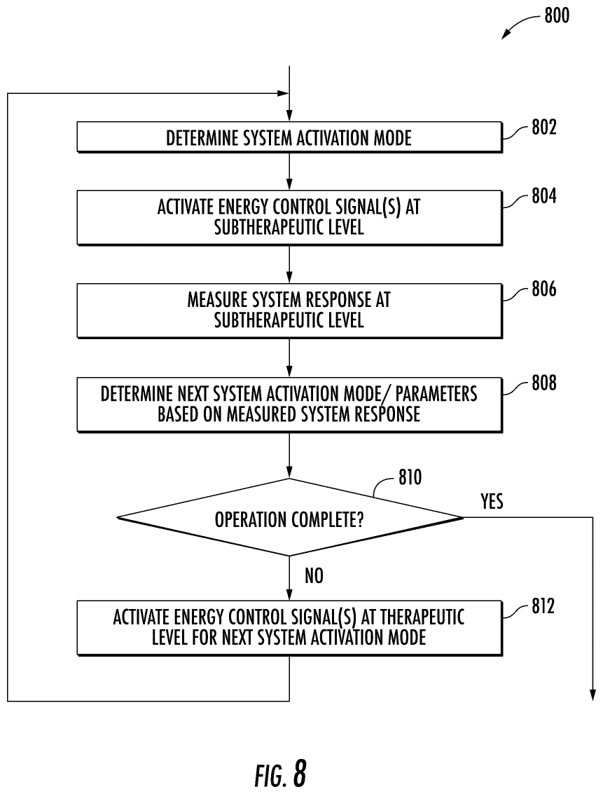

Referring now to FIG. 7, in the illustrative embodiment, the surgical instrument 102 includes a control circuit 700. The control circuit 700 includes a controller 702 and the trigger assembly 150, which cooperate to provide ultrasonic energy to the harmonic blade 130 of the jaw assembly 122 of the end effector 120 and/or RF energy to the RF electrodes 500 of the jaw assembly 122, depending on the operation modes of the surgical instrument 102 as discussed above. In other embodiments, however, the control circuit 700 may include additional or other electronic devices and/or circuit.

The controller 702 may be embodied as any type of controller, functional block, digital logic, or other component, device, circuitry, or collection thereof capable of performing the functions described herein. In illustrative embodiment, the controller 702 includes a processor 704, a memory 706, and an input/output (I/O) subsystem 708. The processor 704 may be embodied as any type of processor capable of performing the functions described herein. For example, the processor 704 may be embodied as a single or multi-core processor(s), digital signal processor, microcontroller, or other processor or processing/controlling circuit. Similarly, the memory 706 may be embodied as any type of volatile and/or non-volatile memory or data storage capable of performing the functions described herein. In operation, the memory 706 may store various data and software used during operation of the control circuit 700 such as executable firmware or software, programs, libraries, and drivers, which may be executed or otherwise used by the processor 704.

The processor 704 and memory 706 are communicatively coupled to other components of the control circuit 700 via the I/O subsystem 708, which may be embodied as circuitry and/or components to facilitate input/output operations between the controller 702 (e.g., the processor 704 and the memory 706) and the other components of the control circuit 700. For example, the I/O subsystem 708 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 708 may form a portion of a system-on-a-chip (SoC) and be incorporated, along with the processor 704 and the memory 706, and other components of the surgical instrument 102, on a single integrated circuit chip. Additionally, in some embodiments, the memory 706, or portions of the memory 706, may be incorporated into the processor 704.

During operation, as discussed above, the controller 702 is configured to control activation of an ultrasonic mode and/or an electro-surgical/RF mode of the surgical instrument 102. To do so, the controller 702 may monitor for activation of the primary trigger 152 and/or one or more activation switches 154 of the trigger assembly 150. In response to activation of the appropriate trigger 152 or switch 154, the controller 702 controls the transducer 104 to generate the ultrasonic energy, which is propagated to the harmonic blade 130 via the ultrasonic waveguide 670. Additionally or alternatively, in response to activation of a corresponding switch 154 of the trigger assembly 150, the controller 702 may be configured to supply an amount of RF energy, via the electro-surgical generator module 164 to the RF electrodes 500 via interconnections 710. It should be appreciated that, although the transducer 104 and the generator 106 are shown as separate components from the energy-based surgical instrument 102 in FIGS. 1 and 7, the transducer 104 and/or the generator 106 may be incorporated into the surgical instrument 102 in other embodiments.

Referring now to FIG. 8, a method 800 for controlling an energy-based surgical instrument 102 is shown. The method 800 may be executed by the controller 702, the generator 106, and/or one or more other microcontrollers or other control elements of the system 100. The method 800 begins in block 802, in which the control element determines a system activation mode for the surgical instrument 102. The system activation mode may include an energy modality (e.g., RF, ultrasound, combined RF and ultrasound, etc.), a surgical operation or firing to be performed with the surgical instrument 102 (e.g., seal, transect, seal and transect, etc.), and/or a sub-operation or phase (e.g., heating, sensing, sealing, cutting, etc.).

In block 804, the control element activates one or more energy control signals at a subtherapeutic level. The subtherapeutic level may be a lower power or energy level that does not cause coagulation, transection, or other therapeutic actions in tissue. The subtherapeutic level may cause other responses in the tissue, such as subtherapeutic heating. The subtherapeutic control may activate ultrasound energy, RF energy, or combined ultrasound and RF energy at the subtherapeutic level.

In block 806, the control element measures a system response at the subtherapeutic level. For example, the control element may measure tissue impedance, acoustic impedance, frequency shift, phase shift, or other responses to application of the subtherapeutic signal.

In block 808, the control element determines a next system activation mode and/or parameters based on the measured system response. For example, the control element may determine whether to switch energy modalities (e.g., from RF to ultrasound, from ultrasound to RF, from a single modality to a combined modality, or other change in energy modality). As another example, the control element may determine whether to change sub-operation or phase, e.g., from pre-heating to sealing, from sealing to transecting, or other change in sub-operation. As another example, the control element may determine one or more parameters for application of therapeutic levels of energy, such as setpoint, amplitude, frequency, crest factor (CF), or other parameters. As yet another example, the control element may determine that the surgical operation (e.g., sealing and/or transecting tissue) has been completed.

In block 810, the control element checks whether the present surgical operation or firing has been completed. If so, the method 800 is completed. The method 800 may be executed again in response to subsequent surgical firings. If the surgical operation is not complete, the method 800 advances to block 812.

In block 812, the control element activates one or more energy control signals at a therapeutic level for the next system activation mode determined as described above. For example, the control element may activate ultrasound and/or RF energy at a setpoint determined as described above or otherwise cause activation of the surgical instrument 102. After activation, the method 800 may loop back to block 802 to continue performing subtherapeutic measurement and control of therapeutic energy application.

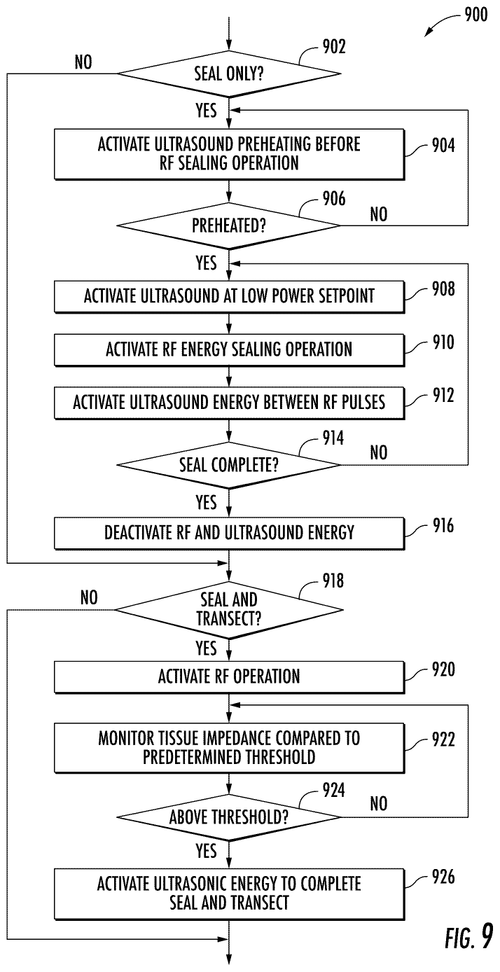

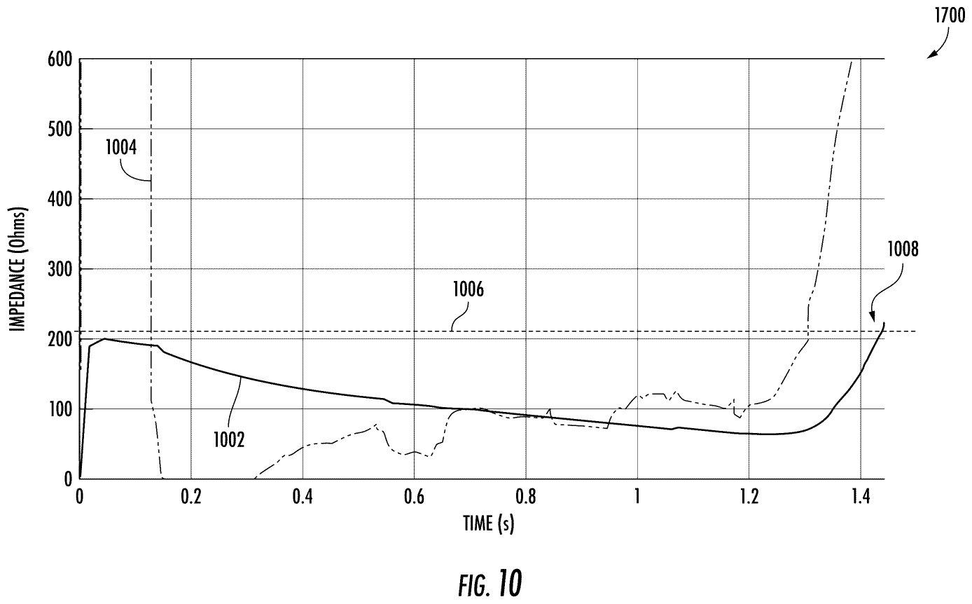

Additionally or alternatively, in some embodiments the control element may perform the operations of the method 800 in a different order and/or in a different combination. Further, in some embodiments the control element may perform additional or different operations and/or make additional or different measurements. Illustrative examples of control operations that may be performed in connection with the surgical instrument 102 are described further below in connection with FIGS. 9-11.

Referring now to FIG. 9, a method 900 for controlling an energy-based surgical instrument 102 is shown. The method 900 may be executed by the controller 702, the generator 106, and/or one or more other microcontrollers or other control elements of the system 100. The method 900 may be executed, for example, in connection with combined RF and ultrasound operation of the surgical instrument 102. The method 900 begins in block 902, in which the control element determines whether to perform a seal-only operation. The control element may determine, for example, whether a surgeon or other user of the surgical instrument 102 has requested a seal-only operation with the combined RF and ultrasound modalities using one or more buttons or other user interface controls of the surgical instrument 102. Using both RF and ultrasound modalities during a seal-only mode may improve hemostasis. If the control element determines not to perform a seal-only operation, the method 900 branches ahead to block 918, described below. If a seal-only-operation is performed, the method 900 advances to block 904.

In block 904, the control element activates ultrasound preheating before an RF sealing operation. For example, ultrasonic energy may be activated, and a Controlled Thermal Management (CTM) algorithm may drive temperature of the ultrasonic blade 130 to a low temperature such as 120° C. to preheat tissue before activating RF energy. Ultrasonic energy may stop once RF energy starts, to allow a seal without transection. Additionally or alternatively, as described further below, ultrasonic energy may continue at a low current setpoint to maintain some heating without transection.

In block 906, the control element determines whether the preheating operation has completed. For example, the control element may determine whether the ultrasonic blade 130 and/or the patient's tissue has reached a desired temperature (e.g., 120° C.). If not, the method 900 loops back to block 904 to continue ultrasonic preheating. If the preheating operation is completed, the method 900 advances to block 908.

In block 908, the control element activates ultrasonic energy at a low power level. For example, the control element may provide ultrasonic energy to the ultrasonic blade 130 at a relatively low current setpoint. The ultrasonic energy level is selected to provide tissue sealing without performing transection. The low-power ultrasonic energy delivery continues during an RF sealing operation, as described further below. Running the ultrasonic energy at a low current set point during an RF firing may provide additional sealing without transection.

In block 910, the control element activates a radio frequency (RF) energy sealing operation. The RF sealing operation delivers RF energy to one or more electrodes 500 of the end effector 120 to perform tissue (e.g., blood vessel) sealing. As described above, the RF energy may be delivered contemporaneously with the low-power delivery of ultrasonic energy. In some embodiments, the RF sealing operation is a pulsing operation that delivers RF energy in discrete pulses.

In block 912, the control element activates ultrasonic energy at a sealing level between RF pulses of the RF sealing operation. The ultrasonic energy is delivered at an energy level that performs sealing without transection. The energy level of the ultrasonic energy delivered between pulses may be higher than the low-power ultrasonic energy that is delivered during delivery of the RF energy. Accordingly, with a pulsing RF algorithm, the ultrasonic energy may be activated briefly between pulses for additional sealing without transection.

In block 914, the control element determines whether the seal operation is complete. The control element may, for example, determine whether a predetermined sealing operation time has elapsed. The predetermined sealing operation time may be shorter than the time required for an RF-only sealing operation. Of course, in other embodiments, the control element may determine whether a seal is complete based on other criteria, such as tissue impedance or other measured values. If the seal is not complete, the method 900 loops back to block 908 to continue activating ultrasonic and RF energy. If the seal is complete, the method 900 advances to block 916, in which the control element deactivates delivery of the RF energy and the ultrasonic energy (i., e, sealing energy, pre-heating energy, and low-power energy). After deactivating energy delivery, the method 900 advances to block 918.

Although described and illustrated in FIG. 9 as performing pre-heating, activating ultrasound at a low-power setpoint, and performing a pulsed RF sealing operation with higher energy ultrasound delivered between pulses, it should be understood that in some embodiments the control element may perform some or all of those operations independently or in combination. For example, in an embodiment, the control element may activate low-power ultrasound as described in block 908 in combination with the RF sealing operation, but without delivering higher-powered ultrasound energy between RF pulses as described in block 912. As another example, in an embodiment, the control element may activate higher power ultrasound between RF pulses as described in block 912 without applying the low-power setpoint ultrasound as described in block 908. In some embodiments, one or both of those operations may be performed with ultrasonic preheating as described in block 904. Of course, other combinations of those operations may be performed in other embodiments.

In block 918, the control element determines whether to perform a seal and transect operation. The control element may determine, for example, whether a surgeon or other user of the surgical instrument 102 has requested a seal-and-transect operation with the combined RF and ultrasound modalities using one or more buttons or other user interface controls of the surgical instrument 102. Performing both RF and ultrasound modalities during a seal and transect may maintain the hemostasis of an RF firing followed by ultrasound with an improved cycle time. If a seal-and-transect operation is not performed, the method 900 is completed. If a seal-and-transect operation is performed, the method 900 advances to block 920.

In block 920, the control element activates an RF seal-and-transect operation. During the RF seal-and-transect operation, the control element delivers RF energy to one or more electrodes 500 of the end effector 120 to perform tissue (e.g., blood vessel) sealing followed by transection.

In block 922 the control element monitors tissue impedance compared to a predetermined threshold. The control element may, for example, measure tissue impedance during delivery of RF energy, between pulses of RF energy, or otherwise measure impedance during the RF seal-and-transect operation. During the RF seal-and-transect operation, upon application of RF energy, the tissue impedance may rise to a particular level and then fall gradually. As the seal progresses, the tissue impedance starts to rise again. The predetermined threshold impedance corresponds to a level of impedance when the seal is nearly complete. Additionally or alternatively, in some embodiments the control element may monitor the change in impedance (i.e., slope), and determine when the change in impedance exceeds a predetermined threshold.

In block 924, the control element checks whether the tissue impedance exceeds the predetermined threshold impedance. If not, the method 900 loops back to block 922 to continue monitoring tissue impedance during application of RF energy. If the tissue impedance exceeds the predetermined threshold impedance, the method 900 advances to block 926.

In block 926, the control element activates an ultrasound transection operation. The control element causes delivery of ultrasonic energy to the ultrasonic blade 130, which completes the seal and transects the tissue. In some embodiments, the ultrasound energy may be activated in combination with the RF energy to complete the seal and transect the tissue. Additionally or alternatively, the RF energy delivery may stop, and only ultrasound energy completes the seal and transection. Accordingly, a combination button or mode on the device 102 may allow for faster seal and cut cycle times while maintaining burst pressure from the RF followed by combination seal. After activating the ultrasonic energy to complete the seal and transection operation, the method 900 is completed.

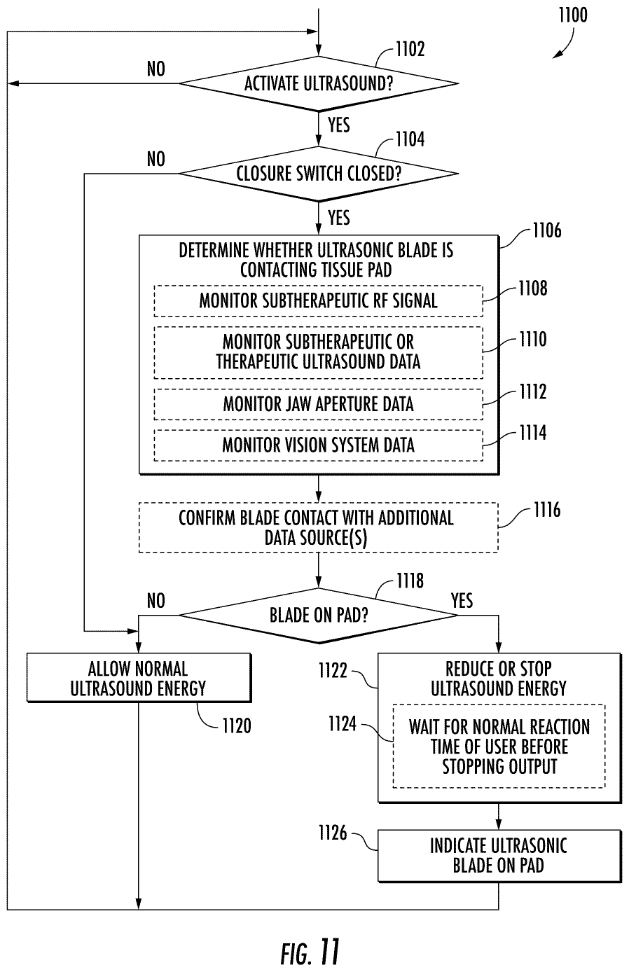

Referring now to FIG. 10, chart 1000 illustrates operation of a seal and transect operation with combined RF and ultrasound modalities. Curve 1002 of the chart 1000 illustrates measured tissue impedance, and curve 1004 illustrates impedance slope that may be measured during an RF seal operation as described above. Line 1006 illustrates a predetermined impedance threshold corresponding to when the seal is nearly completed, and is illustratively about 200 Ω. As shown, the impedance 1002 rises above the predetermined threshold 1006 at point 1008. Accordingly, in the illustrative embodiment, the ultrasound energy modality starts at point 1008.

Referring now to FIG. 11, a method 1100 for controlling an energy-based surgical instrument 102 is shown. The method 1100 may be executed by the controller 702, the generator 106, and/or one or more other microcontrollers or other control elements of the system 100. The method 1100 may be executed, for example, in connection with combined RF and ultrasound operation of the surgical instrument 102. The method 1100 begins in block 1102, in which the control element determines whether to activate ultrasound energy. For example, the control element may determine whether the surgeon or other user has requested activation of ultrasound energy, for example using one or more buttons or other user interface controls of the surgical instrument 102. If the control element determines not to activate ultrasound energy, the method 1100 loops back to block 1102 to continue monitoring for requests to activate ultrasound energy. If the control element determines to activate ultrasound energy, the method 1100 advances to block 1104.

In block 1104, the control element determines whether a closure switch is closed. A full closure switch may be located in the instrument 102 to tell the generator when the user has fully or mostly compressed the closure handle. If the full closure switch is not engaged, the user can activate ultrasonic energy at full power, as described further below. Accordingly, if the closure switch is not closed, the method 1100 branches ahead to block 1120, described below. If the closure switch is closed, the method 1100 advances to block 1106.

In block 1106, the control element determines whether the ultrasonic blade 130 contacts the tissue pad. Detecting the blade 130 contacting the pad may be based on subtherapeutic RF data before or during a harmonic, pulse, subtherapeutic ultrasonic data, therapeutic ultrasonic data, jaw aperture data, vision system data, or a combination thereof. In some embodiments, in block 1108 the control element may monitor a subtherapeutic RF signal. For example, when the device 102 jaws are fully clamped, as indicated by a full closure switch, and the harmonic button is pressed, subtherapeutic RF energy can be delivered before and/or during the ultrasonic activation. Continuing that example, if the electrical impedance during the subtherapeutic energy delivery rises above an “open circuit” threshold, the ultrasonic blade is considered to be touching the tissue pad and energy to the ultrasonic modality is reduced or shut off as described below.

In some embodiments, in block 1110 the control element may monitor subtherapeutic or therapeutic ultrasound data. If subtherapeutic ultrasonic is running before a therapeutic ultrasonic firing, the harmonic impedance, impedance slope, impedance phase, natural frequency, natural frequency slope, or combination thereof can be used to determine when the blade is directly contacting the tissue pad. For therapeutic levels of ultrasonic energy, ultrasonic impedance generally starts high, lowers as tissue is cut, and rises again as the cut completes. Accordingly, if ultrasonic impedance or impedance slope reach a positive threshold after the impedance has reduced during the firing, the ultrasonic blade can be considered in contact with the tissue pad.

In some embodiments, in block 1112 the control element may monitor jaw aperture data. Even small amounts of tissue props jaws of the end effector open. The jaw aperture lowers as the ultrasonic modality cuts through tissue. If the jaw aperture drops to or below a value representative of full closure without tissue in the jaw, then the control element could determine that the ultrasonic blade is in contact with the tissue pad. The expected jaw aperture at full jaw closure may be adjusted as the device is used, for example to account for erosion of the tissue pad. Additionally or alternatively, the user could be prompted to close the jaws fully outside the patient to reconfirm the measurement at full closure.

In some embodiments, in block 1114 the control element may monitor vision system data. The vision system may be programmed to detect contact between the ultrasonic blade and the tissue pad.

In some embodiments, in block 1116, the control element may confirm the blade contact using one or more additional data sources. The control element may confirm the blade contact using multiple of the data sources described above in connection with blocks 1108-1114. For example, if subtherapeutic RF detects the ultrasonic blade 130 on the tissue pad, the control element may consult vision system data to confirm blade is contacting pad before reducing or shutting off energy to the ultrasonic modality. Additionally or alternatively, the control element may combine data features from multiple data sources to detect contact between the ultrasonic blade 130 and the tissue pad. For example, the control element may combine jaw aperture, subtherapeutic RF impedance, and therapeutic harmonic data.

In block 1118, the control element determines whether the blade 130 is in contact with the tissue pad. If so, the method 1100 branches ahead to block 1122, described below. If the blade 130 is not in contact with the tissue pad, the method 1100 advances to block 1120.

In block 1120, the control element allows application of normal ultrasound energy. After allowing application of normal ultrasound energy, the method 1100 loops back to block 1102 to continue monitoring for the blade 130 contacting the pad.

Referring again to block 1118, if the blade 130 is in contact with the tissue pad, the method 1100 branches to block 1122, in which the control element reduces or stops application of ultrasound energy. If subtherapeutic energy was started before the ultrasonic firing, the energy delivered by the ultrasonic modality can be reduced at the start of the firing or the firing can be prevented before it starts. In some embodiments, in block 1124 the control element 1124 may wait for a normal reaction time of the user before stopping the ultrasound output. For example, if the ultrasonic energy is reduced, a wait time corresponding to the normal reaction time of the user can be introduced before completely shutting off the ultrasonic power.

In block 1126, the control element indicates that the ultrasonic blade 130 is contacting the tissue pad. In some embodiments, a visual warning may be generated on a screen of the generator or overlaid on a surgical video stream to indicate to the surgeon that the blade 130 is being activated on the tissue pad, or that the ultrasonic firing has ended. In some embodiments, an audible tone may be emitted from the generator 106 to indicate that the surgeon is activating the blade 130 on the tissue pad.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. A method for controlling a surgical instrument, the method comprising:
activating, by a control element, a pulsed radio frequency (RF) sealing operation with an electrode of an end effector of the surgical instrument, wherein the pulsed RF sealing operation delivers RF energy in a series of pulses; and
activating, by the control element, an ultrasound sealing operation with an ultrasonic blade of the end effector between pulses of the pulsed RF sealing operation, wherein the ultrasound sealing operation delivers ultrasonic energy at a first energy level.

2. The method of claim 1, further comprising activating, by the control element, ultrasonic energy at a second energy level with the ultrasonic blade during activation of the pulsed RF sealing operation, wherein the second energy level is lower than the first energy level.

3. The method of claim 1, further comprising activating, by the control element, ultrasonic energy at a second energy level with the ultrasonic blade before activation of the pulsed RF sealing operation to preheat tissue to a predetermined temperature.

4. The method of claim 3, further comprising activating, by the control element, ultrasonic energy at a third energy level with the ultrasonic blade during activation of the pulsed RF sealing operation and after preheating of the tissue, wherein the third energy level is lower than the first energy level.

5. The method of claim 1, further comprising:
determining, by the control element, a requested combined RF and ultrasound operation, wherein the requested combined RF and ultrasound operation comprises a seal operation or a seal and transection operation; and
performing, by the control element, the requested combined RF and ultrasound operation with the surgical instrument;
wherein when the requested combined RF and ultrasound operation comprises the seal operation, performing the requested combined RF and ultrasound operation with the surgical instrument comprises activating the pulsed RF sealing operation and activating the ultrasound sealing operation.

6. The method of claim 5, wherein when the requested combined RF and ultrasound operation comprises the seal and transect operation, performing the requested combined RF and ultrasound operation with the surgical instrument comprises:
activating, by the control element, an RF seal and transection operation with the electrode of the end effector, wherein the RF seal and transection operation delivers RF energy to tissue of a patient;
measuring, by the control element, a tissue impedance while activating the RF seal and transection operation;
determining, by the control element, whether the tissue impedance exceeds a predetermined threshold impedance; and
activating, by the control element, an ultrasound transection operation with the ultrasonic blade of the end effector in response to determining that the tissue impedance exceeds the predetermined threshold, wherein the ultrasound transection operation delivers ultrasonic energy to the tissue of the patient.

7. A method for controlling a surgical instrument, the method comprising:
activating, by a control element, a radio frequency (RF) seal and transection operation with an electrode of an end effector of the surgical instrument, wherein the RF seal and transection operation delivers RF energy to tissue of a patient;
measuring, by the control element, a tissue impedance while activating the RF seal and transection operation;
determining, by the control element, whether the tissue impedance exceeds a predetermined threshold impedance; and
activating, by the control element, an ultrasound transection operation with an ultrasonic blade of the end effector in response to determining that the tissue impedance exceeds the predetermined threshold, wherein the ultrasound transection operation delivers ultrasonic energy to the tissue of the patient.

8. The method of claim 7, further comprising deactivating, by the control element, the RF seal and transection operation in response to activating the ultrasound transection operation.

9. The method of claim 7, further comprising continuing, by the control element, the RF seal and transection operation in response to activating the ultrasound transection operation.

10. A method for controlling a surgical instrument, the method comprising:
determining, by a control element, whether an ultrasonic blade of an end effector of the surgical instrument is contacting a tissue pad of the end effector of the surgical instrument;
allowing, by the control element, ultrasonic energy delivery at a first energy level in response to determining that the ultrasonic blade is not contacting the tissue pad; and
allowing, by the control element, ultrasonic energy delivery at a second energy level lower than the first energy level in response to determining that the ultrasonic blade is contacting the tissue pad.

11. The method of claim 10, further comprising:
determining, by the control element, whether a closure switch of the surgical instrument indicates that a jaw clamp of the end effector is closed;
wherein allowing the ultrasonic energy delivery at the first energy level further comprises allowing the ultrasonic energy delivery at the first energy level in response to determining that the closure switch indicates that the jaw clamp is not closed or determining that the ultrasonic blade is not contacting the tissue pad; and
wherein allowing the ultrasonic energy delivery at the second energy level comprises allowing the ultrasonic energy delivery at the second energy level in response to determining that the closure switch indicates that the jaw clamp is closed and determining that the ultrasonic blade is contacting the tissue pad.

12. The method of claim 10, further comprising indicating, by the control element, that the ultrasonic blade is contacting the tissue pad in response to determining that the ultrasonic blade is contacting the tissue pad.

13. The method of claim 10, wherein determining whether the ultrasonic blade is contacting the tissue pad comprises:
applying subtherapeutic radio frequency (RF) energy with an electrode of the end effector;
measuring electrical impedance while applying the subtherapeutic RF energy; and
determining whether the electrical impedance exceeds a first threshold, wherein the first threshold is indicative of an open circuit.

14. The method of claim 10, wherein determining whether the ultrasonic blade is contacting the tissue pad comprises:
applying therapeutic or subtherapeutic ultrasonic energy with the ultrasonic blade;
measuring ultrasonic impedance or ultrasonic impedance slope while applying the therapeutic or subtherapeutic ultrasonic energy; and
determining whether the ultrasonic impedance or ultrasonic impedance slope exceeds a positive threshold after the ultrasonic impedance has reduced.

15. The method of claim 10, wherein determining whether the ultrasonic blade is contacting the tissue pad comprises monitoring jaw aperture data, monitoring force on the jaws, or monitoring vision system data.

16. The method of claim 10, wherein determining whether the ultrasonic blade is contacting the tissue pad comprises monitoring a plurality of data sources, wherein the plurality of data sources comprises a subtherapeutic RF signal, a subtherapeutic ultrasound signal, a therapeutic ultrasound signal, jaw aperture data, jaw force data, or vision system data.

17. The method of claim 10, wherein allowing the ultrasonic energy delivery at the second energy level comprises reducing ultrasound energy delivery to the ultrasonic blade from the first level to the second level in response to determining that the ultrasonic blade is contacting the tissue pad.

18. The method of claim 17, further comprising:
waiting, by the control element, a predetermined time after reducing the ultrasound energy delivery, wherein the predetermined time corresponds to a predetermined user reaction time; and
stopping, by the control element, ultrasound energy delivery after waiting the predetermined time.

19. The method of claim 10, wherein the second energy level comprises a zero level, and wherein allowing the ultrasonic energy delivery at the second energy level comprises preventing ultrasonic energy delivery.

20. The method of claim 19, further comprising:
receiving, by the control element, a command to activate the ultrasonic energy delivery to the ultrasonic blade of the end effector;
wherein determining whether the ultrasonic blade is contacting the tissue pad comprises determining whether the ultrasonic blade is contacting the tissue pad in response to receiving the command to activate the ultrasonic energy delivery.
